(19) United States
(12) Patent Application Publication
(10) Pub. No.: US 2013/0112020 A1
(43) Pub. Date: May 9, 2013

(54) BIOMATERIAL TEST APPARATUS INCLUDING A DISC

(71) Applicant: SAMSUNG ELECTRONICS CO., LTD., Suwon-si (KR)

(72) Inventors: Young Goun Lee, Seoul (KR); Chung Ung Kim, Yongin-si (KR); Beom Seok Lee, Hwaseong-si (KR)

(73) Assignee: SAMSUNG ELECTRONICS CO., LTD., Suwon-si (KR)

(21) Appl. No.: 13/667,401

(22) Filed: Nov. 2, 2012

(30) Foreign Application Priority Data
Nov. 3, 2011 (KR) 10-2011-0113654

(51) Int. Cl. G01N 33/00

(52) U.S. Cl. 73/866

(57) ABSTRACT

A biomaterial test apparatus includes a disc having a top surface and a bottom surface, and including at least one biomaterial test site, and a magnetic substance disposed in the disc, and including a protruding portion that protrudes toward the bottom surface of the disc.

### CROSS-REFERENCE TO RELATED APPLICATION

This application claims priority from Korean Patent Application No. 10-2011-0113654, filed on Nov. 3, 2011 in the Korean Intellectual Property Office, the disclosure of which is incorporated herein by reference.

### BACKGROUND

1. Field

Apparatuses and articles of manufacture consistent with exemplary embodiments relate to a biomaterial test apparatus including a disc which may be positioned at a desired position when rotation is stopped.

2. Description of the Related Art

A biomaterial test apparatus may include a disc provided with a biomaterial test site. Upon testing, the disc is first rotated at a high speed, and then the biomaterial test site is photographed after stopping the disc, to determine whether or not a biomaterial has defects.

In the related art, to move the disc to a desired position after the rotation of disc is stopped at an arbitrary position, a magnet may be provided in the disc. As a magnetic substance is horizontally moved to below the disc, the magnet inside the disc may be pulled to the magnetic substance below the disc.

Both the magnet inside the disc and the magnetic substance below the disc may have a circular plate shape.

Although a circular plate shaped magnetic substance, such as a metal, may be provided in the disc instead of the circular plate shaped magnet, this may make it difficult for centers of the magnetic substances below and inside the disc to completely coincide with each other. This misalignment occurs whenever the rotating disc is stopped, causing different images to be acquired whenever the biomaterial test site on the disc is photographed after stopping the disc.

Accordingly, there is a need for a technique to locate the disc at a desired position when the rotating disc is stopped.

### SUMMARY

One or more exemplary embodiments provide a disc and a biomaterial test apparatus including the disc, in which the disc may be accurately positioned.

In accordance with an aspect of an embodiment, there is provided a biomaterial test apparatus including a disc having a top surface and a bottom surface, and including at least one biomaterial test site, and a magnetic substance disposed in the disc body and having a protruding portion that protrudes toward the bottom surface of the disc.

The protruding portion of the magnetic substance may have a lower surface facing the bottom surface of the disc and an upper surface opposite the lower surface, and a flange may be formed at a periphery of the upper surface of the magnetic substance so as to be parallel to the lower surface.

The flange may include a first flange formed at a part of the periphery of the upper surface, and a second flange formed at the periphery of the upper surface at the opposite position of the first flange.

The magnetic substance may be positioned in the disc such that the flange coincides with a rotating direction of the disc.

The disc body may include an upper disc having a top surface and a bottom surface having a first recessed seat in which the flange is seated, and a lower disc having a bottom surface and a top surface having a second recessed seat in which the protruding portion is seated.

The lower surface of the magnetic substance may be opened or closed, and the upper surface may be open. The protruding portion may internally define an empty space.

The interior of the protruding portion may therefore be filled with the same or a different magnetic substance.

A cross sectional area of the protruding portion may decrease toward the bottom surface of the disc.

The magnetic substance contained in the disc may include one or more magnetic substances, and the one or more magnetic substances may be arranged in the disc such that distances between the respective magnetic substances and the center of the disc have different values.

The magnetic substance may include at least one of a magnetic metal, an alloy containing the magnetic metal, and a magnet.

The magnetic metal may include at least one of iron oxide, chromium oxide, ferrite, iron, nickel, and cobalt.

In accordance with an aspect of another exemplary embodiment, there is provided a biomaterial test apparatus includes a disc having a top surface and a bottom surface, one or more biomaterial test sites and a magnetic substance disposed within the disc, and a rotational drive unit to rotate the disc or stop rotation of the disc. The magnetic substance includes a protruding portion that protrudes downward from within the disc towards the bottom surface of the disc.

The protruding portion of the magnetic substance may have a lower surface facing downward towards the bottom surface of the disc, and an upper surface opposite the lower surface. A flange may be formed at the periphery of the upper surface so as to be parallel to the lower surface.

The flange may include a first flange formed at a part of the periphery of the upper surface, and a second flange formed at the periphery of the upper surface at the opposite position of the first flange.

The magnetic substance may be positioned within the disc such that the flange coincides with a rotating direction of the disc.

The flange may be formed along the periphery of the upper surface.

The disc may include an upper disc having a top surface and a first surface, and disposed within the first surface is a first seat into which the flange of the magnetic substance is seated, and a lower disc having a bottom surface and a second surface, and disposed within the second surface is a second seat into which the protruding portion of the magnetic substance is seated.

The lower surface may of the protruding portion of the magnetic substance be a closed surface, and the upper surface of the protruding portion of the magnetic substance may be open, and the protruding portion may internally define an empty space.

The interior of the protruding portion may be filled with the magnetic substance.

A cross sectional area of the protruding portion may decrease toward the bottom surface of the disc.

A plurality of magnetic substances may be disposed in the disc, and the plurality of magnetic substances may be arranged within the disc such that distances between the respective magnetic substances and the center of the disc have different values.

The magnetic substance may include at least one of a magnetic metal, an alloy containing the magnetic metal, and a magnet.

The magnetic metal may include at least one of iron oxide, chromium oxide, ferrite, iron, nickel, and cobalt.

### BRIEF DESCRIPTION OF THE DRAWINGS

The above and/or other aspects will become apparent and more readily appreciated from the following description of exemplary embodiments, taken in conjunction with the accompanying drawings of which:

FIG. 1 is a schematic view illustrating a test apparatus according to an exemplary embodiment;

FIG. 2 is a perspective view illustrating the appearance of a magnetic substance according to an exemplary embodiment;

FIG. 3 is a plan view and a side view of the magnetic substance shown in FIG. 2;

FIG. 4 is a sectional view illustrating the magnetic substance shown in FIG. 2 inserted in a disc; and

FIGS. 5A and 5B are views illustrating a position correction method of the disc performed in the test apparatus according to an exemplary embodiment.

### DETAILED DESCRIPTION

Reference will now be made in detail to the embodiments, examples of which are illustrated in the accompanying drawings, wherein like reference numerals refer to like elements throughout.

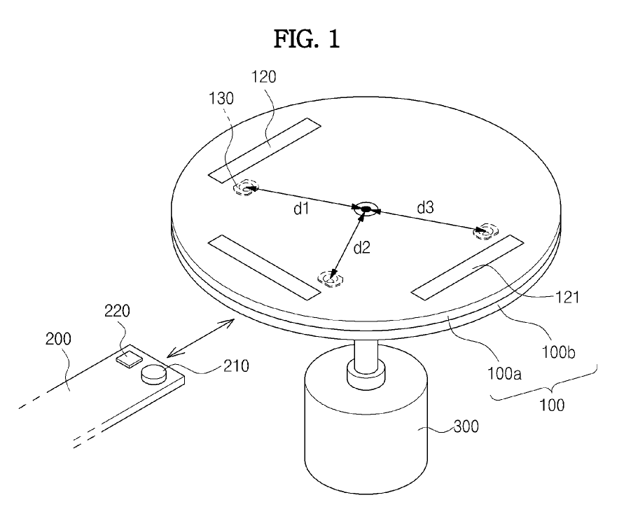

FIG. 1 is a schematic view illustrating a test apparatus according to an exemplary embodiment.

A test apparatus serves to perform analysis or testing of a variety of samples. There may be various test apparatuses based on the kinds of samples to be analyzed.

Although examples of a sample may include deoxyribonucleic acid (DNA), oligo-nucleotide, ribonucleic acid (RNA), peptide nucleic acid (PNA), ligand, receptor, antigen, antibody, milk, urine, saliva, hair, crop sample, meat sample, avian sample, cattle sample, processed food sample, mouth swab, tissue sample, semen, protein, or other biomaterials, the disclosure is not limited thereto. Also, although examples of an object to be analyzed may include protein, antigen, antibody, DNA, RNA, oligo-nucleotide, or receptor, the disclosure is not limited thereto. For example, when a sample is urine, examples of an object to be analyzed may include blood, glucose, ascorbic acid, ketone, protein, sugar, urobilinogen, and bilirubin.

As illustrated in FIG. 1, the test apparatus according to the exemplary embodiment includes a disc 100, a rotation drive unit 300 to rotate the disc 100, and a sled 200 located below the disc 100.

The disc 100 is a disc-shaped platform having a top surface and a bottom surface, and is provided with one or more biomaterial test sites 120 and 121. Although not illustrated in the drawings, each biomaterial test site 120 or 121 may include a plurality of strips having different brightnesses.

In an exemplary embodiment, the biomaterial test sites 120 and 121 may be formed on the disc 100 by printing. Strips of the biomaterial test sites 120 and 121 may be printed uniformly on the disc 100. To this end, the biomaterial test sites 120 and 121 may be printed using silk screen printing. However, this is given by way of example, and the inventive concept is not limited thereto.

At least one magnetic substance 130 is provided in the disc 100. More specifically, the disc 100 may include an upper disc 100a having an top surface and a bottom surface, and a lower disc 100b having a top surface and a bottom surface. The upper disc 100a and the lower disc 100b are stacked such that the bottom surface of the upper disc 100a is disposed on the top surface of the lower disc 100b. The bottom surface of the upper disc 100a and the top surface of the lower disc 100b are respectively provided with recessed seats or areas into which the magnetic substance 130 is seated. In this way, the magnetic substance 130 may be located in the disc 100 such that an upper portion of the magnetic substance 130 is disposed in the recessed seat of the upper disc 100a and a lower portion of the magnetic substance 100 is disposed in the recessed seat of the lower disc 100b.

A plurality of magnetic substances 130 may be provided to correspond to the biomaterial test sites 120 and 121 in a one to one ratio, and may be located adjacent to the respective biomaterial test sites 120 and 121. The respective magnetic substances 130 may be arranged at different distances from the center of the disc 100. For example, FIG. 1 illustrates an exemplary embodiment in which three magnetic substances are provided in the disc 100. In this case, distances between the respective magnetic substances 130 and the center of the disc 100 are referred to as d1, d2, and d3, and have a relationship of d1>d2>d3.

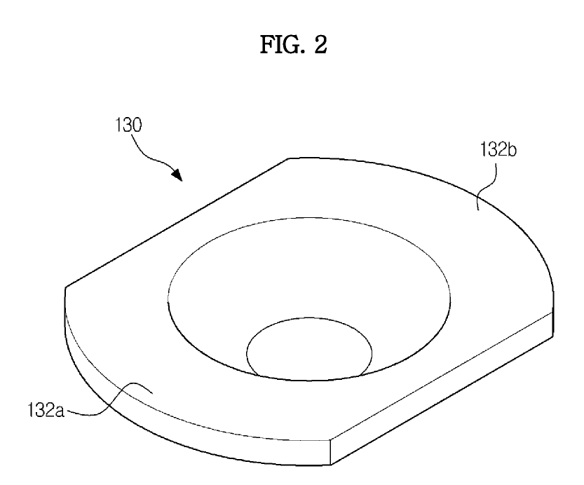

In the exemplary embodiment, the magnetic substance 130 may be include, for example, a magnetic metal, an alloy containing the magnetic metal, and a magnet. Examples of the magnetic metal may include, but are not limited to, a ferromagnetic metal, such as iron oxide, chromium oxide, ferrite, iron (Fe), nickel (Ni), and cobalt (Co). The magnetic substance 130 may have various shapes, and a more detailed description with respect to the shape of the magnetic substance 130 will hereinafter be described with reference to FIGS. 2 to 4.

The sled 200 is provided with a circular plate shaped magnet 210 and a camera 220 to photograph the biomaterial test sites 120 and 121. The sled 200 is located below the disc 100 and is horizontally movable with respect to the disc 100.

A horizontal movement distance of the sled 200 may be determined based on which one of the biomaterial test sites 120 and 121 on the disc 100 is to be photographed. That is, if the biomaterial test site 121 corresponding to the magnetic substance 130 that is located closest to the center of the disc 100 is to be photographed, a movement distance of the sled 200 may be correspondingly increased. If the biomaterial test site 120 corresponding to the magnetic substance 130 that is located farthest from the center of the disc 100 is to be photographed, a movement distance of the sled 200 may be correspondingly decreased.

Next, the magnetic substance according to an exemplary embodiment will be described with reference to FIGS. 2 to 4.

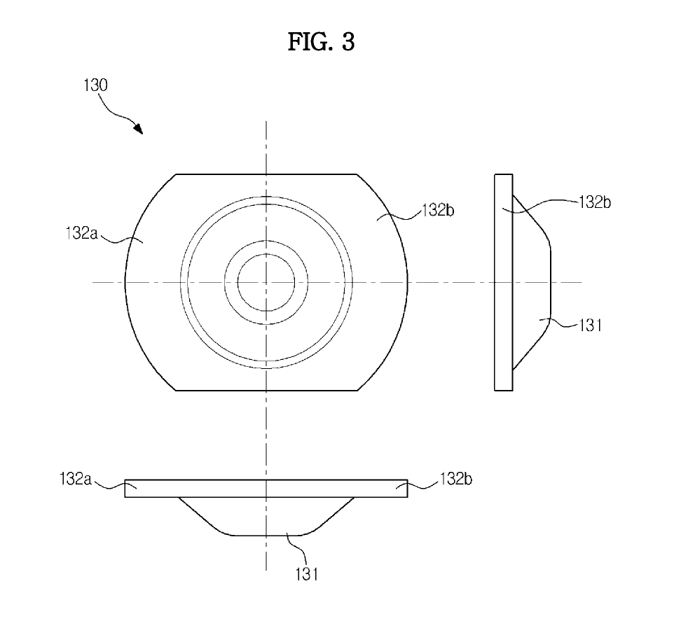

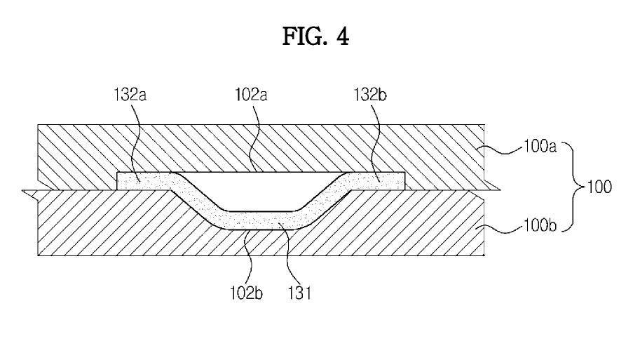

FIG. 2 is a perspective view illustrating the appearance of the magnetic substance according to the exemplary embodiment, FIG. 3 is a plan view and a side view of the magnetic substance shown in FIG. 2, and FIG. 4 is a sectional view illustrating the magnetic substance shown in FIG. 2 inserted in the disc 100.

Referring to FIGS. 2 to 3, the magnetic substance 130 includes a downwardly protruding portion 131.

The protruding portion 131 has opposite upper and lower surfaces. The lower surface is a surface that is oriented to face the magnet 210 of the sled 200 located below the disc 100. The protruding portion 131 is configured such that a cross section thereof is tapered from the upper surface to the lower surface. In one example, the protruding portion 131 may have a conical, triangular pyramidal, or quadrangular pyramidal shape. In another example, the protruding portion 131 may take the form of a cone, triangular pyramid, or quadrangular pyramid having a stepped sloping side. However, the disclosure is not limited thereto, and any other shapes may be included in the disclosure so long as the cross section thereof is tapered from the upper surface to the lower surface. Providing the protruding portion 131 with the downwardly tapered cross section may increase attractive force between the protruding portion 131 of the magnetic substance 130 and the magnet 210 of the sled 200.

Provided at the periphery of the upper surface of the magnetic substance 130 are flanges 132a and 132b parallel and opposite to each other. The flanges 132a and 132b serve to increase attraction with the magnet 210 of the sled 200. The flanges 132a and 132b also serve to secure the magnetic substance 130 at a predetermined position within the disc 100. Through this configuration in which the first and second flanges 132a and 132b are located at opposite positions to one another, interaction between the respective magnetic substances 130 provided on the disc 100 may be reduced, as compared to the case in which the magnetic substances provided on the disc 100 are configured such that a flange is formed throughout the periphery of the upper surface of each magnetic substance.

The lower surface of the protruding portion 131 a closed surface, and the upper surface of the protruding portion 131 is open, such that an empty space is defined within the protruding portion 131.

With reference now to FIG. 4, the bottom surface of the upper disc 100a is provided with a first recessed seat 102a, which has a shape corresponding to the upper portion of the magnetic substance 130, i.e., has a shape corresponding to the flanges 132a and 132b. The top surface of the lower disc 100b coming into contact with the bottom surface of the upper disc 100a is provided with a second seat recessed 102b at a position corresponding to a position of the first recessed seat 102a of the upper disc 100a. The second recessed seat 102b has a shape corresponding to the lower portion of the magnetic substance 130, i.e. has a shape corresponding to the protruding portion 131. In this way, as the bottom surface of the upper disc 100a and the top surface of the lower disc 100b are coupled to each other, after the flanges 132a and 132b of the magnetic substance 130 have been seated in the first recessed seat 102a of the upper disc 100a and the protruding portion 131 of the magnetic substance 130 has been seated in the second recessed seat 102b of the lower disc 100b, the magnetic substance 130 is mounted within the disc 100.

In various embodiments, a plurality of magnetic substances 130 are provided in the disc 100, wherein the bottom surface of the upper disc 100a is provided with a number of first recessed seats 102a corresponding to the number of the magnetic substances 130, and the top surface of the lower disc 100b is provided with a number of second recessed seats 102b corresponding to the number of the magnetic substances 130.

The magnetic substances 130 as described above may be mass-produced using, for example, drawing or punching.

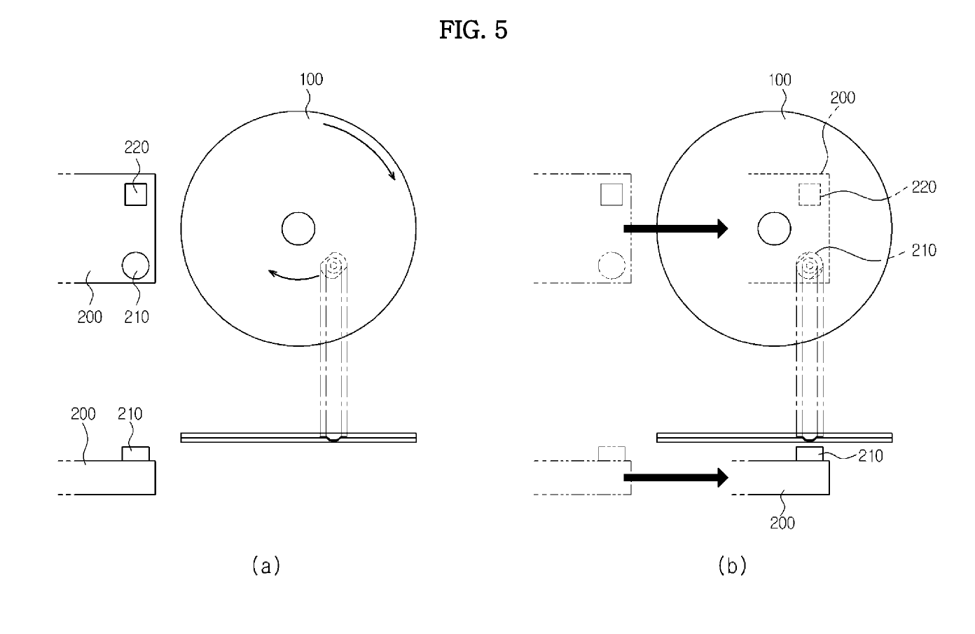

FIG. 5 is a view illustrating a position correction method of the disc performed in the test apparatus according to an exemplary embodiment.

When the disc 100 is rotated, the magnetic substance(s) 130 located in the disc 100 are rotated along with the disc 100. During rotation of the disc 100, the sled 200 is located at the outside of the disc 100 (i.e., away from the rotational path of the disc 100), as shown in FIG. 5(a).

Thereafter, when it is desired to photograph the specific biomaterial test site 120 on the disc 100, the rotating speed of the disc 100 is reduced, and simultaneously, the sled 200 is horizontally moved so as to be located below the disc 100 as shown in FIG. 5(b). In this case, a movement distance of the sled 200 may be determined based on a distance between the center of the disc 100 and the magnetic substance 130 adjacent to the corresponding biomaterial test site 120 to be photographed.

Once the sled 200 has been positioned below the disc 100, rotation of the disc 100 stops via attraction between the magnet 210 of the sled 200 and the magnetic substance 130 of the disc 100.

When the disc 100 stops at a desired position, the camera 220 of the sled 200 photographs the corresponding biomaterial test site 120.

As is apparent from the above description, according to the exemplary embodiment, a disc included in a biomaterial test apparatus contains a ferromagnetic substance, a cross sectional area of which decreases toward a magnet of a sled. As compared to installing a circular plate shaped magnetic substance inside the disc, the disc may be more accurately positioned when rotation is stopped.

Further, providing the disc with the ferromagnetic substance rather than a magnet may reduce production costs of the biomaterial test apparatus.

Furthermore, the ferromagnetic substance provided in the disc may be mass produced, resulting in reduction in production costs of the biomaterial test apparatus.

Although the disc and the biomaterial test apparatus including the same according to the exemplary embodiment have been shown and described, it would be appreciated by those skilled in the art that changes may be made in the embodiment without departing from the principles and spirit of the invention, the scope of which is defined in the claims and their equivalents. Also, although the above described embodiment describes the case in which an empty space is defined in the protruding portion, the interior of the protruding portion may be filled with the magnetic substance.

What is claimed is:

1. A biomaterial test apparatus comprising:
a disc having a top surface and a bottom surface, and including at least one biomaterial test site; and
a magnetic substance disposed in the disc, and including a protruding portion that protrudes downward towards the bottom surface of the disc.

2. The biomaterial test apparatus according to claim 1,
wherein the protruding portion of the magnetic substance has a lower surface facing the bottom surface of the disc and an upper surface opposite the lower surface, and
wherein the magnetic substance further includes a flange is formed at a periphery of the upper surface so as to be parallel to the lower surface.

3. The biomaterial test apparatus according to claim 2, wherein the flange includes:
a first flange formed at a part of the periphery of the upper surface; and
a second flange formed at the periphery of the upper surface at the opposite position of the first flange.

4. The biomaterial test apparatus according to claim 3, wherein the magnetic substance is positioned in the disc such that the flange coincides with a rotating direction of the disc.

5. The biomaterial test apparatus according to claim 2, wherein the flange is formed along the periphery of the upper surface.

6. The biomaterial test apparatus according to claim 2, wherein the disc comprises:
an upper disc having a top surface, and a bottom surface having a first recessed seat in which the flange of the magnetic substance is seated; and
a lower disc having a bottom surface, and a top surface having a second recessed seat in which the protruding portion of the magnetic substance is seated, and
wherein the upper disc is disposed on the lower disc so that the bottom surface of the upper disc contacts the top surface of the lower disc.

7. The biomaterial test apparatus according to claim 2,
wherein the lower surface of the protruding portion of the magnetic substance is a closed surface and the upper surface of the protruding portion of the magnetic substance is open, and
wherein the protruding portion internally defines an empty space.

8. The biomaterial test apparatus according to claim 2, wherein an interior of the protruding portion is filled with the magnetic substance.

9. The biomaterial test apparatus according to claim 1, wherein a cross sectional area of the protruding portion decreases toward the bottom surface of the disc.

10. The biomaterial test apparatus according to claim 1, further comprising:
a plurality of magnetic substances disposed in the disc,
wherein the plurality of magnetic substances are arranged in the disc such that distances between the respective magnetic substances and a center of the disc have different values.

11. The biomaterial test apparatus according to claim 1, wherein the magnetic substance includes at least one of a magnetic metal, an alloy containing the magnetic metal, and a magnet.

12. The biomaterial test apparatus according to claim 11, wherein the magnetic metal includes at least one of iron oxide, chromium oxide, ferrite, iron, nickel, and cobalt.

13. A biomaterial test apparatus comprising:
a disc having a top surface and a bottom surface, and including at least one biomaterial test site;
a magnetic substance disposed in the disc, and including a protruding portion that protrudes downward towards the bottom surface of the disc; and
a rotation drive unit configured to rotate the disc or stop rotation of the disc.

14. The biomaterial test apparatus according to claim 13,
wherein the protruding portion of the magnetic substance has a lower surface facing the bottom surface of the disc and an upper surface opposite the lower surface, and
wherein the magnetic substance further includes a flange is formed at a periphery of the upper surface so as to be parallel to the lower surface.

15. The biomaterial test apparatus according to claim 14, wherein the flange includes:
a first flange formed at a part of the periphery of the upper surface; and
a second flange formed at the periphery of the upper surface at the opposite position of the first flange.

16. The biomaterial test apparatus according to claim 15, wherein the magnetic substance is positioned in the disc such that the flange coincides with a rotating direction of the disc.

17. The biomaterial test apparatus according to claim 14, wherein the flange is formed along the periphery of the upper surface.

18. The biomaterial test apparatus according to claim 14, wherein the disc includes:
an upper disc having a top surface, and a bottom surface having a first recessed seat in which the flange of the magnetic substance is seated; and
a lower disc having a bottom surface, and a top surface having a second recessed seat in which the protruding portion of the magnetic substance is seated, and
wherein the upper disc is disposed on the lower disc so that the bottom surface of the upper disc contacts the top surface of the lower disc.

19. The biomaterial test apparatus according to claim 14,
wherein the lower surface of the protruding portion of the magnetic substance is a closed surface and the upper surface of the protruding portion of the magnetic substance is open, and
wherein the protruding portion internally defines an empty space.

20. The biomaterial test apparatus according to claim 14, wherein an interior of the protruding portion is filled with the magnetic substance.

21. The biomaterial test apparatus according to claim 13, wherein a cross sectional area of the protruding portion decreases toward the bottom surface of the disc.

22. The biomaterial test apparatus according to claim 13, further comprising
a plurality of magnetic substances disposed in the disc, and
wherein the plurality magnetic substances are arranged in the disc such that distances between the respective magnetic substances and a center of the disc have different values.

23. The biomaterial test apparatus according to claim 13, wherein the magnetic substance includes at least one of a magnetic metal, an alloy containing the magnetic metal, and a magnet.

24. The biomaterial test apparatus according to claim 23, wherein the magnetic metal includes at least one of iron oxide, chromium oxide, ferrite, iron, nickel, and cobalt.
